(19) United States
(12) Patent Application Publication
(10) Pub. No.: US 2011/0196374 A1
(43) Pub. Date: Aug. 11, 2011

(54) CUTTING ELEMENT FOR A TOOL FOR SHAPING A BONE SURFACE, AND TOOL PROVIDED WITH SAME

(76) Inventors: Michel Porte, Le Blanc-Mesnil (FR); Lévon Doursounian, Paris (FR)

(21) Appl. No.: 12/736,192

(22) PCT Filed: Mar. 17, 2009
(86) PCT No.: PCT/FR2009/050438

§ 371 Date: Apr. 26, 2011

(30) Foreign Application Priority Data
Mar. 17, 2008 (FR) 08 51701

(51) Int. Cl. A61B 17/16

(52) U.S. Cl. 606/80

(57) ABSTRACT

A cutting element (1) for a tool for shaping a bone surface includes a plurality of elements that form metal blades (2), each having at least one cutting edge (5, 16); a substrate (4) made of a different material than that of the blade elements (2); said blade elements (2) being integrated into the substrate (4) during the manufacture of the cutting element and being tubular in shape and featuring a cutting flange (5) at least one of the ends thereof, and in that they comprise a longitudinal axis (XX) having an incline of an angle (α) relative to the substrate surface (4), this angle (α) being between 35 and 45°.

A tool for shaping a bone surface equipped with such a cutting element.

The invention concerns the field of surgical instruments, and more particularly tools designed to give a precise shape to a bone surface, for example before placing a prosthesis.

### BACKGROUND

It must be understood that the term “bone surface”, in this description, refers both to a natural external surface (for example a cotyle of the pelvis receiving a femoral head or the glenoid cavity receiving the humeral head) and an inner surface to which the surgeon has access following sectioning of a bone part (for example the medullary cavity of a long bone such as a femur or a humerus, which becomes accessible after sectioning the femoral or humeral head to place the femoral or humeral element of a hip or shoulder prosthesis).

During placement of a hip prosthesis, before placing the cotyloid implant the surgeon must prepare the surface of the natural cotyle, so as to grant it the shape best suited to maintenance of the implant. This step is particularly important when using implants that are placed purely by impaction (“press fit” technique), without using a sealing material, and for which the shape of the corrected cotyloid cavity must be strictly adapted to that of the rear face of the implant to ensure optimal maintenance thereof after its placement.

This shaping of the cotyle is done by the surgeon using a “cotyle drill,” i.e. an instrument that can be made to rotate and has, at its end, a cutting element that can be removed so that it can be replaced when necessary. This cutting element has a generally hemispherical shape, and has protrusions forming blades that cut the wall of the cotyle and correct it by giving it the desired shape and dimensions. This cutting element is usually made of a single material, such as stainless steel.

This cutting element has the drawback of being expensive to make, due to the fact that it is made from a costly material and requires complex machining during the last step of its production. Furthermore, it gradually wears during its use and thus becomes less effective. It must therefore be replaced regularly, unless it can be re-machined to restore its cutting capacities. Such re-machining is difficult, however, cannot be done on the surgical site, and makes the tool unusable for a certain period of time if a spare part is not immediately available. And over time, this re-machining can end up causing unacceptable modifications of its dimensions. Above all, between each use of the element, it is necessary to clean, decontaminate and sterilize it carefully, and preserve that sterile nature all the way to the operating block. Management of these cutting elements by the hospital, in order to ensure their constant availability under suitable conditions, is therefore complex. If it is faulty, it can lead to canceling an operation at the last minute.

### SUMMARY OF THE INVENTION

It is an object of the present invention to provide cutting elements for tools for shaping bone surfaces, in particular cotyles, with a lower production cost than existing cutting elements, as well as optimal effectiveness.

The present invention provides a cutting element for a tool for shaping a bone surface, characterized in that it includes:

a plurality of elements that form metal blades, each having at least one cutting edge;

a substrate made of a different material than that of the blade elements;

said blade elements being integrated into the substrate during the manufacture of the cutting element, and being tubular in shape and featuring a cutting flange at least one of the ends thereof, and in that they comprise a longitudinal axis (XX) having an incline of an angle (α) relative to the substrate surface, this angle (α) being between 35 and 45°.

Said substrate can be made of polymer, plastic or elastomer.

Said substrate can have been made by over molding around said blade elements.

Said substrate can have been made by blowing, and have had the previously-machined blade elements mechanically installed there.

Said substrate can have an inner cavity.

Said blade elements can have protrusions and/or hollows on their outer surfaces favoring their hooking to the substrate.

The general shape of the cutting element can be hemispherical or practically spherical.

The present invention also provides a tool for shaping a bone surface, characterized in that it includes a removable cutting element of the aforementioned type at its end.

As will have been understood, in the invention, the cutting element of a tool for shaping a bone surface is made not in the form of a single piece, machined so as to give it the protrusions forming blades ensuring cutting of the bone surface, but in the form of a multi-material piece. The cutting of the bone is done by a series of pieces forming protrusions on the outer surface of the element, and each having a cutting edge forming a blade to that end. These pieces are normally made from a metal material, identical or comparable to those used to make the traditional single-piece cutting elements, and are integrated, for example by over molding, with a base material forming the rest of the element. This base material must be rigid enough to ensure maintenance of the cutting pieces, but can also be of a less expensive type than that of the cutting parts, for example a polymer such as PMMA or a plastic, or even an elastomer.

According to the invention, these cutting parts are formed by tubular elements having a cutting flange forming a blade, and whereof the longitudinal axis is inclined in relation to the line perpendicular to the surface of the base material of the element by an angle between 35 and 45°.

The cutting elements can be made through mass production, by bar turning, and using a relatively small amount of material, which reduces the overall cost of the raw materials used to manufacture the element and the costs related to the production of the metal parts of the element. As for the base material making up the support of these cutting elements, its material cost is low, and the over molding or blowing that makes it possible to optimally integrate the cutting elements therein is a simple and quick operation, only requiring molds and an injection machine not posing any particular design and implementation problems.

Moreover, the reduced cost of making these elements makes it possible to design them as single-use sterile tools, which has the advantages of:

guaranteeing a tool of optimal and consistent quality for each use, without requiring periodic re-machining to maintain that quality;

eliminating the cleaning, decontamination and sterilization done between two uses of the typical cutting elements; one need only, after producing the element according to the invention, sterilize and package it, the packaging only being opened at the last minute on the operating site; after use, the element is simply discarded; this greatly facilitates management of the material for the operating site because it is thus easy to determine before an operation whether the appropriate material is available and in good working order, and to be sure that it is completely sterile.

In order to further limit production costs and improve the overall economic balance of this production and use of the device according to the invention, it is quite possible to partially recycle the used elements by destroying their substrate material, for example by heating or passage through a solvent, and recovering the cutting elements. These are then returned to a metallurgist, and they can be melted out, in particular to produce the component material of new cutting elements.

### BRIEF DESCRIPTION OF THE DRAWINGS

The invention will be better understood upon reading the following description, provided in reference to the following appended figures:

FIG. 1 shows a front view of an example of a device according to the invention, in this case a generally hemispherical head designed to be mounted on a tool for shaping a cotyle;

FIG. 2 shows a detail of the head of FIG. 1 in cross-section along II-II;

FIG. 3 shows this same head in a diving perspective bottom view;

### DETAILED DESCRIPTION

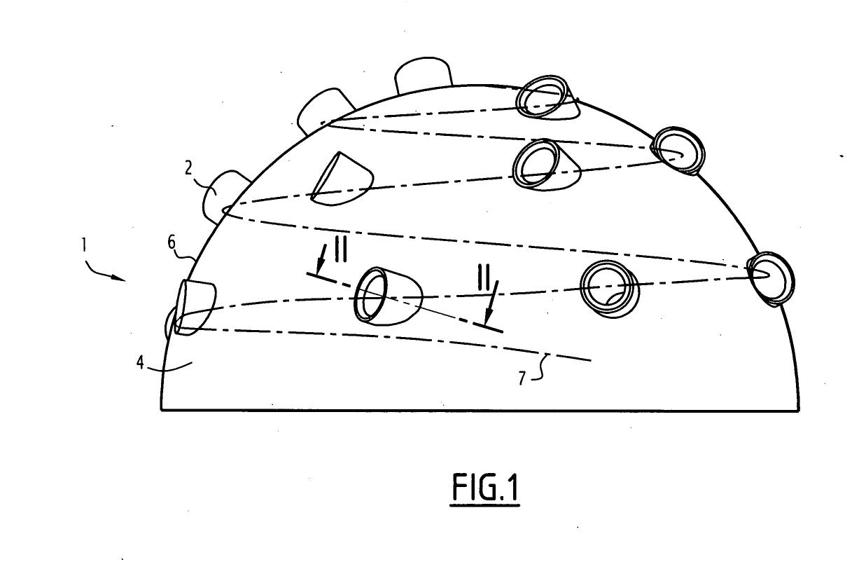

An example of a device according to the invention shown in FIGS. 1 to 3 is a cutting element 1 designed to be mounted at the end of a driving member, to shape a pelvic cotyloid cavity or a shoulder blade glenoid cavity. According to the invention, it includes:

a series of elements forming blades 2 made of a metal material, e.g. stainless steel, which, in the illustrated example, are generally tubular cylindrical, i.e. they have a longitudinal cavity 3 that passes all the way through them;

and a rigid support 4 made of polymer or a plastic material, e.g. PMMA or PET, generally hemispherical in the illustrated example, in which said blade elements are integrated and integrally anchored.

This support 4 has means (not shown) which, traditionally, allow it to be removably fastened to the end of a tool owing to which the surgeon can give the device a rotational movement. This means is adapted to the specific tool for which the cutting element 1 is designed. In particular, the hemispherical portion of the substrate 4 can be extended by a short cylindrical portion that, in certain cases, facilitates gripping of the support 4 by the tool. This tool, which can be put in motion either solely manually or also using motorized means, is of a type known in itself and does not need to be described in more detail and illustrated.

If the substrate 4 is made from a transparent plastic material, the surgeon can see the appearance of the cut surface during the operation, without having to interrupt the operation and remove the shaping tool, which is an additional advantage in relation to a completely metal cutting element.

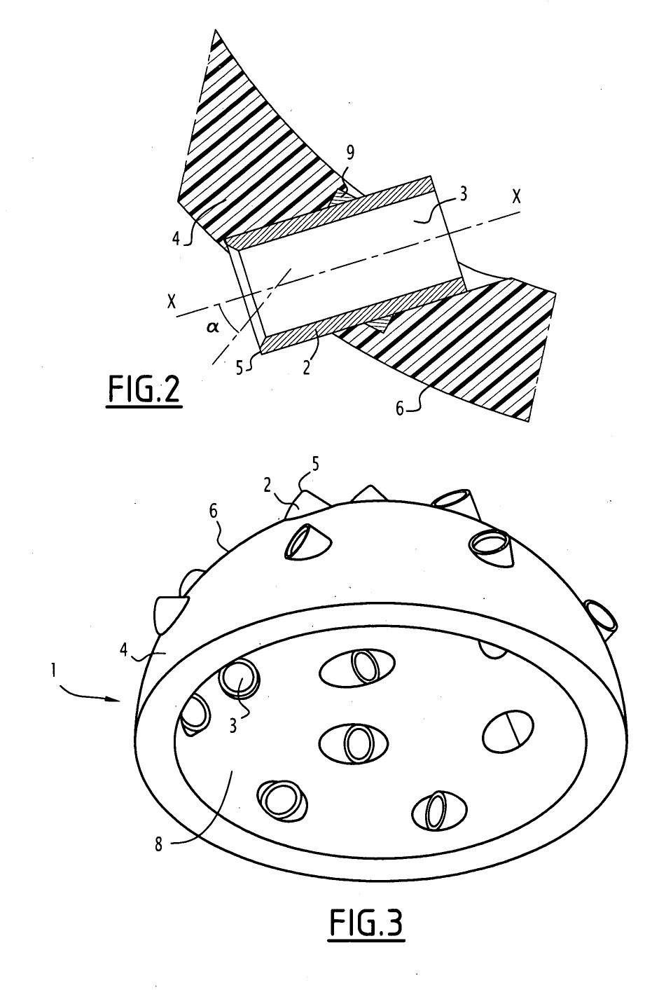

As shown in particular in FIG. 2, the blade elements 2 are configured on the periphery of one of their ends so as to have a cutting flange 5, forming the functional part of the blade element 2 and capable of cutting the bone wall of the patient's cotyloid cavity when the cutting element 1 is placed in the cavity and set in rotation.

The blade elements 2 are arranged in the substrate 4 such that:

their cutting flanges 5 emerge from the outer surface 6 of the substrate 4 on at least one portion of their perimeter, defining an envelope thereof substantially in the form of a hemispherical cap whereof the curvature corresponds to the curvature the surgeon wishes to grant the cotyloid cavity of the treated patient;

their longitudinal axes X-X are oriented so that the cutting flanges 5 can attack the bone wall from a cutting angle granting them good operating effectiveness; this cutting angle is defined by the angle α formed by axis X-X with the line perpendicular to the surface of the substrate 4, and is, according to the invention, between 35 and 45° for optimal cutting effectiveness; preferably this angle α is the same for all of the cutting elements 2, in order to ensure good homogeneity of the shaping of the cavity;

the longitudinal axes X-X of the various blade elements 2 intersect the outer surface 6 of the substrate along a general helical line 7, and are numerous enough that during the cutting operation, the entire surface of the cotyloid cavity can be attacked by the cutting flanges 5 and have, at the end of the operation, as regular a spherical surface as possible, allowing the insertion of the cotyloid implant of the hip or shoulder prosthesis and its maintenance under good conditions.

As stated, one feature of the invention lies in the fact that only the functional parts of the cutting element 1 are made of a metal material that is costly to prepare and machine, the part 4 supporting them being made from an inexpensive material.

One important advantage of the tubular configuration of the blade elements 2 (that is also not necessarily cylindrical, but could be tapered, inter alia) is that if, as in the illustrated example, it is coupled to a substrate 4 itself having, after over molding, an inner cavity 8 left at least partially free after fastening of the element 1 on the tool for shaping the cotyloid cavity, the bone shavings resulting from the shaping can be evacuated through the cavities 3 of the blade elements 2. They are then caught in the cavity 8 of the substrate 4 and do not hinder the progress of the rest of the shaping. It can also be provided that during the operation, they can be removed from the cavity 8 of the substrate 4 by a suction device connected to the shaping tool.

A simple and effective method for making the cutting element 1 according to the invention consists of machining, by bar turning, the blade elements 2 and arranging them on a substrate giving them the desired locations and orientations. An over molding of the plastic material forming the substrate 4 is then done on them, so as to integrate them there. This can be done entirely traditionally by a person skilled in the art, using an injection machine including a suitably shaped mold. It is simply necessary to prevent the plastic material from penetrating the cavities 3 of the blade elements 2 and blocking them during the over molding, unless the cavities 3 are unblocked after the complete solidification of the substrate 4.

Another method for making the element 1 consists of making the substrate 4 by blowing of a polymer material (such as PET), then mechanically installing the previously-machined blade elements 2 there.

To ensure good anchoring of the blade elements 2 in the substrate 4, it is recommended to give them, on their outer surface, protrusions and/or hollows, around or in which the plastic material forming the substrate 4 solidifies. In the illustrated example (see FIG. 2), an annular protrusion 9 with a triangular section has been formed on the outer surface of each blade element 2. This prevents the blade elements 2 from sliding in the substrate 4 along their longitudinal axis X-X. One could also provide other elements, for example protrusions or longitudinal splines, that would prevent rotational sliding of the blades.

Advantageously, during production of the blade elements 2, they can be given a completely symmetrical shape, in particular by forming a cutting flange 5 at each of their edges. This makes it possible to simplify the manufacturing process of the element 1, in that the operators or machines placing the blade elements 2 on their substrate before over molding or blowing of the substrate 4 do not need to worry about the direction in which the blade elements 2 must be placed.

Another embodiment of the invention consists of giving the substrate material a certain flexibility, for example by making it from elastomer. In this way, it is possible to facilitate the shaping of the bone surface by making it more gradual, the substrate 4 tending to better fit the initial shape of the surface than if it was made of a very rigid material. For a tool for shaping a cotyloid cavity, a flexible tool can be used in a first step of the shaping to ensure rough cutting thereof.

Another embodiment of the invention, not shown, may include of giving the substrate 4 a non-hemispherical, but practically spherical shape, and arranging the blade elements 2 on either side of the equator of the sphere portion. In this way, the correct shaping of the cotyloid cavity depends less on the incline of the cutting tool, which makes the surgeon's work easier. For example, the blade elements 2 can extend over a 270° angle, as opposed to a maximum of 180° for a hemispherical substrate 4.

The blade elements 2 can for example have a diameter between 0.5 and 5 mm. The substrate 4 can have a diameter between 18 mm (for veterinary medicine) and 65 mm. It goes without saying that the cutting elements of the invention can be used to shape bone surfaces other than cotyles, if one wishes to give them a shape in the form of a sphere portion.

What is claimed is:

1-7. (canceled)

8. A cutting element for a tool for shaping a bone surface, comprising:
a plurality of blade elements being made of metal, each blade element having at least one cutting edge; and
a substrate made of a different material than that of the blade elements, the substrate having a substrate surface;
the blade elements being integrated into the substrate during the manufacture of the cutting element and being tubular in shape, each blade element having a cutting flange at least one of the ends thereof, and having a longitudinal axis having an incline of an angle relative to the substrate surface of between 35 and 45°.

9. The cutting element as recited in claim 8 wherein the substrate is made of polymer, plastic or elastomer.

10. The cutting element as recited in claim 9 wherein the substrate is overmolded around said blade elements.

11. The cutting element as recited in claim 9 wherein the substrate is a blow-formed substrate with the blade elements being previously-machined blade elements mechanically installed in the blow-formed substrate.

12. The cutting element as recited in claim 8 wherein the substrate has an inner cavity.

13. The cutting element as recited in claim 8 wherein the blade elements have protrusions and/or hollows on an outer surface to aid in hooking to the substrate.

14. The cutting element as recited in claim 8 wherein a shape of the cutting element is hemispherical or substantially spherical.

15. A tool for shaping a bone surface comprising, at an end, a removable cutting element as recited in claim 8.
